(19) United States
(12) Patent Application Publication
(10) Pub. No.: US 2014/0373926 A1
(43) Pub. Date: Dec. 25, 2014

(54) REMOTE MONITORING, CONTROL, AND AUTOMATIC ANALYSIS OF WATER SYSTEMS USING INTERNET-BASED SOFTWARE AND DATABASES

(71) Applicant: HydroNovation, Inc., San Francisco, CA (US)

(72) Inventors: Anil D. Jha, San Francisco, CA (US); Ramandeep Mehmi, Livermore, CA (US); Peter Naylor, San Francisco, CA (US)

(73) Assignee: HYDRONOVATION, INC., San Francisco, CA (US)

(21) Appl. No.: 14/375,395

(22) PCT Filed: Jan. 22, 2013
(86) PCT No.: PCT/US2013/022482

§ 371 Date: Jul. 29, 2014

Related U.S. Application Data
(60) Provisional application No. 61/592,307, filed on Jan. 30, 2012.

(51) Int. Cl. G01N 33/18; G01M 99/00; G05D 7/06

(52) U.S. Cl. CPC G01N 33/18; G05D 7/0617; G01M 99/005
USPC 137/2; 702/25; 702/184; 702/182

(57) ABSTRACT

A control system is configured to remotely read data associated with a process system in real time and perform various functions associated with the obtained data. The data may be stored to a database and/or analyzed to provide various operating parameters to one or more remote clients.

### CROSS-REFERENCE TO RELATED APPLICATION

This application is a national stage entry under 35 U.S.C. §371 of PCT application PCT/US2013/022482, filed Jan. 22, 2013, which claims the benefit of priority to U.S. Provisional Application No. 61/592,307 filed Jan. 30, 2012.

### FIELD OF THE INVENTION

The methods and processes disclosed here generally relate to a method and system for remotely monitoring a water treatment system by using a combination of hardware and software techniques.

### SUMMARY

In one aspect, the methods and systems disclosed here provide a method for monitoring and controlling a water treatment system comprising: remotely reading a stream of measured data associated with the water treatment system, performing an analysis of the measured data to determine one or more measured operating parameters associated with the water treatment system, comparing the measured operating parameter to a target operating parameter, producing an output response based on the comparison between the measured operating parameter to the target operating parameter, and storing, on a storage device, an indication of the output response.

In at least one aspect, the method further comprises creating a database comprising the stored indication of the output response. In another aspect, the method further comprises communicating the database to a graphical user interface. In certain aspects, the output response comprises generating a report. In at least one aspect, the report is generated on a periodic basis. In another aspect, the method further comprises communicating the report to at least one remote client. In certain aspects, the report is communicated to the at least one remote client electronically. In at least one aspect, the stream of measured data is remotely read on a periodic basis. In another aspect the method further comprises adjusting a flow rate of a water stream in the water treatment process based at least in part on the output response. In certain aspects, the target operating parameter is based on a user's preference.

Aspects and embodiments of the present disclosure are directed toward a method for monitoring the removal of unwanted species from a water stream in a water treatment system, the method comprising: remotely reading at least one measured operating value related to the removal of unwanted species from the water stream, comparing the at least one measured operating value to a target value, generating an output response based on the comparison between the measured operating value to the target value, and transmitting the output response to at least one remote client.

In certain aspects, the at least one measured operating value is a concentration of unwanted species in the water stream. In another aspect, the water stream is a product water stream. In at least one aspect, the product water stream has been treated by an electrochemical water treatment device. In another aspect, the at least one measured operating value is a remaining cartridge capacity in at least one treatment device.

In one aspect, the methods and systems disclosed here provide a method for remotely monitoring at least one water treatment system, the at least one water treatment system including at least one water stream, the method comprising: remotely reading data related to the removal of unwanted species from the water stream, storing the data related to the removal of unwanted species from the water stream, analyzing the data related to the removal of unwanted species from the water stream, generating an output response based on the analysis of the data related to the removal of the unwanted species from the water stream, and communicating the output response to at least one remote client.

In one aspect, the output response is generated by a control computer. In another aspect, the method further comprises controlling at least one operating parameter of the water treatment system by the at least one remote client based on the output response.

In at least one aspect, the methods and systems disclosed here provide a system for monitoring a water treatment process comprising: a remote interface device in communication with at least one component of the water treatment process, and a controller in communication with the remote interface device and configured to: read data associated with the water treatment process, perform an analysis of the data associated with the water treatment process to determine one or more parameters associated with the water treatment process, compare the one or more parameters associated with the water treatment process to one or more desired parameters, produce an output response based on the comparison of the one or more parameters associated with the water treatment process to the desired parameters, and store an indication of the output response.

These and other objects, along with advantages and features of the systems and methods described herein, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

Non-limiting embodiments of the systems and methods described herein will be conveyed by way of example, and optionally, with reference to the accompanying drawing. In the following description, various embodiments of the systems and methods described herein are outlined with reference to the following drawing, in which:

FIG. 1 is a flow chart illustrating an exemplary control scheme in accordance with one or more embodiments of the systems and methods disclosed here.

### DETAILED DESCRIPTION

The systems and methods described herein are not limited in their application to the details of construction and the arrangement of components set forth in the description or as illustrated in the drawing(s). The systems and methods described herein are capable of other embodiments and of being practiced or of being carried out in various ways. Also, the phraseology and terminology used herein is for the purpose of description and should not be regarded as limiting. The use of “including,” “comprising,” “having,” “containing,” “involving,” “characterized by,” “characterized in that,” and variations thereof, are meant to encompass the items listed thereafter, equivalents thereof, and additional items, as well as alternate embodiments consisting of the items listed thereafter exclusively.

Water that contains hardness species such as calcium and magnesium may be undesirable for some uses, for example, in industrial, commercial, residential, or household applications. Hard water requires more soap and synthetic detergents for home laundry and washing, and contributes to scaling in pipes, boilers and industrial equipment. Hardness is caused by compounds of calcium and magnesium, as well as a variety of other metals, and is primarily a function of the geology of the area where the ground water is located. Water acts as an excellent solvent and readily dissolves minerals it comes in contact with. As water moves through soil and rock, it dissolves very small amounts of minerals and holds them in solution. Calcium and magnesium dissolved in water are the two most common minerals that make water “hard,” although iron, strontium, and manganese may also contribute. The hardness of water is referred to by three types of measurements: grains per gallon (gpg), milligrams per liter (mg/L), or parts per million (ppm). Hardness is usually reported as an equivalent quantity of calcium carbonate (CaCO₃). One grain of hardness equals 17.1 mg/L or 17.1 ppm of hardness. The typical guidelines for a classification of water hardness are: zero to 60 mg/L of calcium carbonate is classified as soft; 61 mg/L to 120 mg/L as moderately hard; 121 mg/L to 180 mg/L as hard; and more than 180 mg/L as very hard.

Ion exchange is the reversible interchange of ions between a solid, (for example, an ion exchange resin) and a liquid (for example, water). Since ion exchange resins act as “chemical sponges,” they are ideally suited for effective removal of contaminants from water and other liquids. Ion exchange technology is often used in water demineralization and softening, wastewater recycling, and other water treatment processes. Ion exchange resins are also used in a variety of specialized applications, for example, chemical processing, pharmaceuticals, mining, and food and beverage processing.

Hard water contains greater than about 60 ppm of calcium carbonate and is often treated prior to use by a water softener. Typically, the water softener is of the rechargeable ion exchange type and is charged with cation resin in the sodium form and anion resin in the chloride form. As water passes through the resin bed, major contributors to hardness, such as calcium and magnesium species, are exchanged for sodium. In this manner, the water can be softened as the concentration of divalent cations and, in particular, calcium and magnesium ions, decreases.

In water softening systems, the hardness ions become ionically bound to oppositely charged ionic species that are mixed on the surface of the ion exchange resin. The ion exchange resin eventually becomes saturated with ionically bound hardness ion species and must be regenerated. Regeneration typically involves replacing the bound hardness species with more soluble ionic species, such as sodium chloride. The hardness species bound on the ion exchange resin are replaced by the sodium ions and the ion exchange resins are ready again for a subsequent water-softening step. However, an equivalent of sodium is added to the treated water for every equivalent of calcium that is removed. Thus, although the water is softened, the hardness is replaced with sodium ions that some consumers may find undesirable. Furthermore, when these ion exchange beds are recharged, the resulting brine must be disposed of and is often discharged to a septic system where the brine becomes available to re-enter the ground water. In some regions, discharge of brine to a domestic septic system or to the environment is regulated or prohibited.

Other methods of softening water include the use of reverse osmosis devices that can supply high purity water. Many reverse osmosis membranes can be fouled by the presence of dissolved materials such as silica, which may often be found in well water.

In desalination systems, salt and other minerals are removed from water. For example, sea water (or salt water from another source) may be desalinated for use as fresh water suitable for human consumption or irrigation. In other instances, salt water is an undesirable by-product of one or more industrial processes, and must be treated to reduce the salt concentration.

The process systems described herein are directed to water treatment or purification systems and methods of providing treated water in industrial, commercial, residential, and household settings. One or more embodiments will be described using water as the fluid but should not be limited as such. For example, where reference is made to treating water, it is believed that other fluids can be treated according to the systems and methods described herein. Moreover, the treatment systems and apparatuses are believed to be applicable in instances where reference is made to a component of the system or to a method that adjusts, modifies, measures or operates on the water or a property of the water. The fluid to be treated may also be a fluid that is a mixture comprising water. The water treatment systems described herein receive water from a source and subsequently pass it through a process system to produce a product stream possessing targeted characteristics.

The water treatment systems described herein may treat water by providing for the addition of hydrogen ions to the water, which contributes to reducing the corrosivity of the treated water. The addition of hydrogen to the water may manifest itself by a detectable increase in dissolved hydrogen or a resulting decrease in the concentration of oxidative species. This may also provide desirable anti-oxidant properties as well. Advantages include, for example, lower volumes of waste water ejected from the system and increased protection of process components, such as, valves, pipes, sensors, and treatment devices from scale formation. Further advantages include the ability to de-scale certain process components and to lower one or more maintenance costs associated with the treatment system.

The water treatment systems described herein may also treat water by controlling the conductivity of the water in one or more components of the process system. For example, the water treatment system may provide liquids, such as water, possessing a low conductivity. The water treatment systems described herein may also treat water by performing a desalination process on high salinity liquid, such as seawater.

A problem encountered with monitoring the performance of a process system remotely is the optimization of the process in real time. This problem is exaggerated when the monitoring requires analyzing the performance of multiple systems in real time. Many process systems require monitoring and analysis to be done physically on site. This is expensive in terms of providing service personnel, retaining continuity of service, and preserving the life of equipment, since the ability to anticipate a malfunction or problem is limited. The lack of ability to monitor real-time process system operating conditions leads to a monitoring response style that is more retro-active, rather than pro-active.

As used herein, the term “monitoring” refers to any activity including recording, observing, evaluating, identifying, detecting, measuring, calculating, and any other action that encompasses test information or data and any other measures for obtaining information concerning an operation, process, process system, and components of a process.

As used herein, the term “control” refers to any device, mechanism, or process that is capable of affecting an operation, process, process system, and components of a process. For example, control may be an operation performed by a computer, mechanical, electronic, user, and any other method used to operate and maintain a process system. The term includes, without limitation, the ability to permit, restrict, or prevent a specified activity within a process, whether it is related to a single dedicated function or a plurality of functions. The operation may include, for example, powering, stopping, guiding, starting, restarting, pausing, regulating, or otherwise influencing one or more components in a process system.

As used herein, the term “remote” refers to the transmission of information, for example, data, control signals, power signals, or other interactions between separated devices or apparatuses, such as a controller or a water treatment device, that are located at some distance from each other. The term “remote” does not imply a particular spatial relationship between the controller and the water treatment device, which may, in various embodiments, be separated by relatively large distances, for example miles or kilometers, or relatively small distances, for example, inches or millimeters.

As used herein, the terms “communicate,” “transmit,” “send,” “report,” “relay,” and the like refer to any type and/or manner of providing, supplying, inputting, or otherwise transmitting data or data sets. Likewise, the terms “read,” “receive,” “collect,” “accept,” and the like refer to receiving data or data sets. The transmission of the data or data sets is may be carried out electronically, including the use of wired electronic methods, wireless electronic methods, or combinations thereof. For example, the transmission of the data or data sets may be performed by a wire or cable, such as one or more USB cables. Electronic transmissions can be carried out by a variety of local or remote electronic transmission methods, such as by using Local or Wide Area Network (LAN or WAN)-based, internet-based, or web-based transmission methods, cable television, or wireless telecommunications networks, or any other suitable local or remote transmission method.

As used herein, the terms “information,” “data,” and “parameter” are broadly construed to comprise signals, (for example, analog signals, digital signals, wireless signals, and the like), states, data (for example, process system data) for providing knowledge, values, events, facts, measures, outcomes, and similar items.

As used herein, the term “process system” may be any conceivable type of process, for example, a water treatment process, a manufacturing process, a steady state or batch process, a chemical process, a material handling process, an energy production process, a waste removal process, a fuel replenishing process, and so forth. In an exemplary embodiment, as will be described in further detail below, the process system may be implemented in the context of a water treatment process.

As used herein, “hardness” refers to a condition that results from the presence of polyvalent cations, typically calcium, magnesium, or other metals, in water, that adversely affects the cleansing capability of the water and the “feel” of the water and may increase scaling potential. Hardness is typically quantified by measuring the concentration of calcium and magnesium species. In certain embodiments, undesirable species can include hardness ion species.

As used herein, the phrases “treatment device” or “purification device” or “apparatus” pertain to any device that can be used to remove or reduce the concentration level of any undesirable species from a fluid to be treated. Examples of suitable treatment apparatuses include, but are not limited to, devices related to ion-exchange resin, reverse osmosis, electrodeionization, electrodialysis, ultrafiltration, microfiltration, pre- or post-treatment devices, and capacitive deionization.

As used herein, the term “treated” in reference to water, fluid, or liquid, refers to low TDS water, low Langelier Saturation Index (LSI) water and/or low conductivity water.

The Langelier Saturation Index (LSI) is a calculated number used to predict the calcium carbonate stability of water. LSI may be calculated according to a standard method, for example, ASTM D 3739. The resulting value indicates whether the water will precipitate, dissolve, or be in equilibrium with calcium carbonate. For LSI>0, water is super saturated and tends to precipitate a scale layer of CaCO₃. For LSI=0 or LSI close to 0, water is saturated (in equilibrium) with CaCO₃. A scale layer of CaCO₃ is neither precipitated nor dissolved. Water quality, changes in temperature, or evaporation could change the index. For LSI<0, water is under saturated and tends to dissolve solid CaCO₃. As used here, low LSI water has an LSI of less than about 2, preferably, less than about 1, and more preferably, less than or about zero.

Electrical conductivity (EC) is a measure of the water's ability to “carry” an electrical current, and, indirectly, a measure of dissolved solids or ions in the water. Pure water has a very low conductivity value (nearly zero); hence, the more dissolved solids and ions occurring in the water, the more electrical current the water is able to conduct. A conductivity probe in conjunction with a temperature sensor is capable of determining the electrical resistance of a liquid. Fresh water usually reflects electrical conductivity in units of micro Siemens (μS/cm). As used here, a low conductivity liquid has a conductivity of less than about 300 μS/cm.

Total Dissolved Solids (TDS) are the total amount of mobile charged ions, including minerals, salts or metals dissolved in a given volume of water, expressed in units of mg per unit volume of water (mg/L), also referred to as parts per million (ppm). TDS is directly related to the purity and quality of water and water purification systems and affects everything that consumes, lives in, or uses water, whether organic or inorganic. The term “dissolved solids” refers to any minerals, salts, metals, cations or anions dissolved in water, and includes anything present in water other than the pure water molecule and suspended solids. In general, the total dissolved solids concentration is the sum of the cations and anions in the water. TDS is based on the electrical conductivity (EC) of water, with pure water having virtually no conductivity.

As used herein, the phrase “electrochemical water treatment device” refers to any number of electrochemical water treatment devices, non-limiting examples including, electrodeionization devices, electrodialysis devices, capacitive deionization devices, and any combination thereof, and may include devices that may be used in accordance with the principles of the systems and methods described herein as long as they are not inconsistent or contrary to the operation of devices and/or the techniques of the systems and methods described herein.

As used herein, the term “softening device” pertains to an ion exchange device that is capable of producing softened water, low LSI water and/or low conductivity water using a salt based technology.

As used herein, the term “desalination device” refers to any number of apparatuses capable of treating seawater or other brackish waters to reduce the amount of salt or other ionized impurities.

As used herein, the term “system yield” also refers to treatment system recovery, meaning the measure of waste versus production. System yield/recovery rates are determined using the following calculation:

System yield=[Product volume/(Waste volume+Product volume)]*100

As used herein, the term “operating parameter” refers to one or more independent variables that can be controlled in a process system. Non-limiting examples include: feed stream properties (for example, hardness, conductivity, pH, TDS, LSI, flow rate, temperature), product stream properties, concentrate stream properties, system yield, product volume, storage system properties (for example, capacity volume, pressure, temperature, and properties associated with the stored fluid), properties related to the performance of an electrochemical water treatment device (including properties related to transmitting electric current to the device), properties related to the performance of an ion exchange device (including properties associated with one or more replaceable cartridges), valve properties, system flow rates, properties related to pre-treatment devices, pump properties, waste stream properties, and any combination thereof.

As used herein, the term “sensor” refers to any kind of device of which some part or portion is capable either of selectively interacting with a species of interest, thereby producing a well-defined and measurable response which is a function of a characteristic or attribute of that species. In addition, the sensor device is capable of responding to a bulk property of a fluid or a total concentration of one or more species in the fluid.

As used herein, the term “remote client” refers to one or more users of the methods and systems disclosed here, for example, service personnel, homeowners, vendor representatives, manufacturers, and the like. In addition, the remote client may refer to hardware and software, for example, a computer system, and may include a computing system(s), such as a stand-alone personal desktop or laptop computer (PC), workstation, personal digital assistant (PDA), or appliance, to name only a few. A remote client may connect to a network via a communication connection, such as a cell networks, cable, or DSL connection via an Internet service provider (ISP) or may connect directly into a LAN, for example, for building an automation system via network connections.

As used herein, the term “real time” refers to the time intervals (seconds, minutes) during which operating system data is taken from one or more components of the process system and distributed to one or more controllers. As used here, “real time” is a relative term, and actually might be tuned or modified by those implementing the system. In addition, delays in the distribution of data may result from private and/or public (e.g., Internet) network traffic and/or because of a user's network access speed, creating slight variants, which are inherent and to some degree expected in the electronic information distribution infrastructure.

As used herein, the term “periodic basis” refers to the time interval chosen by the user, client, or service provider to monitor the performance of the process system. In certain embodiments, the time interval may be, for example, every one second, every 60 seconds, every 30 minutes, every 60 minutes, every 12 hours, every 24 hours, and any combination thereof.

As used herein, the term “unwanted species” refers to any one or more substances, materials, matter, or organism, that is considered to be unnecessary, undesirable or otherwise inhibits the functionality of the systems and methods disclosed here. A species that is considered “unwanted” in one part or component of the treatment process may actually be desirable in another part of the treatment process. Non-limiting examples of unwanted species include dissolved salts or ionic or ionizable species including sodium, chloride, calcium ions, magnesium ions, hydrogen ions, hydroxyl ions, hydroxide ions, carbonates, fluorides, chlorates, bromates, sulfates or other insoluble or semi-soluble species or dissolved gases, bacteria, contaminants, impurities, and any combination thereof.

The water treatment systems disclosed here can have a water storage system in line with at least one or more treatment devices, non-limiting examples including: electrochemical water treatment devices, reverse osmosis devices, electrodialysis devices, ion exchange resin devices, desalination devices, capacitive deionization devices, microfiltration devices, and/or ultrafiltration devices. The liquid contents of the electrochemical water treatment device may be replaced or supplemented with a liquid having a low LSI, thereby inhibiting scale formation. In addition, the liquid having a low LSI can be sent to a storage system.

The water treatment systems described herein may comprise an electrochemical water treatment device. Electrochemical cells for use in water/waste treatment systems are designed to operate by making use of the water electrolysis process wherein, at the anode-water interface, OH—, being present in water due to electrolytic dissociation of water molecules, donates an electron to the anode and can be thereby oxidized to oxygen gas which can be removed from the system. Non-limiting examples of electrochemical water treatment device include electrodeionization devices, reverse osmosis devices, ion-exchange resin beds, electrodialysis devices, capacitive deionization devices, bipolar membrane desalting devices, and any combination thereof.

One potential problem related to electrochemical water treatment processes is the risk of forming insoluble calcium or magnesium deposits. These deposits are formed at conditions of high Ca 2⁺ and/or Mg 2⁺ concentration and at high pH values.

Electrodeionization (EDI) is a process that can be used to demineralize, purify, or treat water by removing ionizable species from liquids using electrically active media and an electrical potential to influence ion transport. The electrically active media may function to alternately collect and discharge ionizable species, or to facilitate the transport of ions continuously by ionic or electronic substitution mechanisms. EDI devices can include media having a permanent or temporary charge, and can be operated to cause electrochemical reactions designed to achieve or enhance performance. These devices may also include electrically active membranes such as semi-permeable ion exchange or bipolar membranes. Non-limiting examples of electrochemical deionization units include electrodialysis (ED), electrodialysis reversal (EDR), electrodeionization (EDI), capacitive deionization, continuous electrodeionization (CEDI), and reversible continuous electrodeionization (RCEDI).

The water treatment systems described herein may further comprise one or more ion exchange devices, for example, a cation exchange device, comprising cation exchange resin. The ion exchange device may also comprise a regenerable resin device. The regenerable device comprises a cartridge containing an ion exchange resin. When the ion exchange material reaches its exhaustion point or is near exhaustion, it may be regenerated, for example, by a strong or weak acid. The water treatment systems described herein may have one or more ion exchange devices positioned either upstream or downstream of one or more electrochemical water treatment devices.

The water treatment systems described herein may comprise a pre-filter device. The pre-filter device may be a preliminary filter or pre-treatment device designed to remove a portion of any undesirable species from the water before the water is further introduced into one or more components of the treatment system. The pre-filter device may also comprise a regenerable or exchangeable cartridge. Non-limiting examples of pre-filter devices include, for example, carbon or charcoal filters that may be used to remove at least a portion of any chlorine, including active chlorine, or any species that may foul or interfere with the operation of any of the components of the treatment system process flow. Additional examples of pre-treatment devices include, but are not limited to, particulate filters, aeration devices, chlorine-reducing filters, ionic exchange devices, mechanical filters, reverse osmosis devices, and any combination thereof. Pre-treatment systems can be positioned anywhere within the treatment system, and therefore may be considered as post-treatment systems, and may comprise several devices, or a number of devices arranged in parallel or in a series.

The water treatment system comprises at least one or more water streams. For example, a feed stream provides or fluidly communicates water from a water source to the treatment system. Non-limiting examples of suitable water sources include potable water sources, for example, municipal water, well water, non-potable water sources, for example, brackish or salt-water, pre-treated semi-pure water, and any combination thereof. The feed stream may contain dissolved salts or ionic or ionizable species including sodium, chloride, chlorine, calcium ions, magnesium ions, carbonates, sulfates or other insoluble or semi-soluble species or dissolved gases, such as silica and carbon dioxide. The feed stream may also contain additives, such as fluoride, chlorate, and bromate species. The water treatment system can also comprise a recirculating concentrate loop and/or a recirculating dilution loop that recirculates through an electrochemical water treatment device. The water treatment system may include a waste stream, comprising discharged water that exits the process system. In certain embodiments, the waste stream and the recirculating concentrate stream may be in fluid communication with each other. The water treatment system also comprises at least one product stream that has been treated by one or more components of the water treatment process and possesses desired characteristics or properties. For example, product water may comprise a hardness of less than 1 gpg.

The water treatment systems described herein comprise one or more fluid control devices, such as pumps, valves, regulators, sensors, pipes, connectors, controllers, power sources, and any combination thereof.

The water treatment system comprises one or more sensors or monitoring devices disposed to measure at least one property of the water or an operating condition of the water treatment system. Non-limiting examples of sensors include composition analyzers, pH sensors, temperature sensors, conductivity sensors, pressure sensors, and flow sensors. In certain embodiments, the sensors provide real-time detection that reads, or otherwise senses, the properties or conditions of interest. Non-limiting examples of sensors suitable for use include optical sensors, magnetic sensors, radio frequency identification (RFID) sensors, Hall effect sensors, and any combination thereof.

The water treatment systems described herein further comprise at least one flowmeter for sensing and/or regulating the flow of fluid. A non-limiting example of a flowmeter suitable for certain aspects of the treatment system includes a Hall effect flowmeter. Other non-limiting examples of flowmeters include mechanical flowmeters, for example, a mechanical-drive Woltman-type turbine flowmeter. The flow regulator may also be a valve that can be intermittently opened and closed according to a predetermined schedule for a predetermined period of time to allow a predetermined volume to flow. The amount or volume of flowing fluid can be adjusted or changed by, for example, changing the frequency the flow regulator is opened and closed, or by changing the duration during which the flow regulator is open or closed. The flow regulator can be controlled or regulated by a controller, through, for example, a signal. For example, a controller can provide a signal, such as a radio, current or a pneumatic signal, to an actuator, with, for example, a motor or diaphragm, that opens, closes, or otherwise redirects fluid through the flow regulator. The flow regulator can also be controlled by demand from one or more users, for example, by directing or regulating the flow of water to one or more outlets for use.

The water treatment system may include a storage system comprising one or more tanks or vessels, and can be positioned in multiple locations throughout the process system. Additionally, each tank or vessel can have several inlets positioned at various locations. Similarly, outlets can be positioned on each vessel at various locations depending on, among other things, the capacity or efficiency of the water treatment device, demand, flow rates within the process system, and the capacity or hold-up of the storage system. The storage system can further comprise various components or elements that perform desirable functions or avoid undesirable consequences. For example, the tanks or vessels may have internal components, such as baffles, that are positioned to disrupt any internal flow currents or areas of stagnation, and/or relief valves, to prevent unwanted pressure.

The water treatment systems described herein may further comprise at least one disinfecting and/or cleaning apparatus components. Such disinfecting or cleaning systems can comprise any apparatus that destroys or renders inactive, at least partially, any microorganisms, such as bacteria, that can accumulate in any component of the treatment system. Examples of cleaning or disinfecting systems include those that can introduce a disinfectant or disinfecting chemical compounds, such as halogens, halogen-donors, acids or bases, as well as systems that expose wetted components of the treatment system to hot water temperatures capable of sanitization. The water treatment system may also include final stage or post treatment systems or subsystems that provide final purification of the fluid prior to delivery. Examples of such post treatment systems include, but are not limited to those that expose the fluid to actinic radiation or ultraviolet radiation, and/or ozone or the removal of undesirable compounds by microfiltration or ultrafiltration.

The process systems disclosed here are at least partially monitored and/or controlled by a control system having one or more controllers. The control system may perform control functions in response to process information received from the process system. For instance, the process information may be provided by one or more sensors configured to detect and/or measure certain parameters of the process system, which may include measurements. In general, such sensors may include measurement devices, transducers, and the like that may produce discrete or analog signals and values representative of various variables of the process system.

Sensors may be coupled to one or more controllers of a control system, and in fact, many such sensors and more than one controller may be provided in the control system. Sensors commonly produce voltage or current outputs that are representative of the sensed variables. The process information may represent “on-process” measurements of various parameters obtained directly from the process, for example, by using the sensors. Additionally, the process information may also include controllable and external operating constraints, as well as user-specified set points. Non-limiting examples of sensors include, for example, potentiometric sensors, amperometric sensors, and optical sensors.

In certain embodiments, the treatment system also includes a controller for adjusting, monitoring, or regulating at least one operating parameter and its components of the treatment system. A controller typically comprises a microprocessor-based device, such as a programmable logic controller (PLC) or a distributed control system that receives or sends input and output signals to one or more components of a treatment system. In certain embodiments, the controller regulates the operating conditions of the treatment system in an open-loop or closed-loop control scheme. For example, the controller, in open-loop control, can provide signals to the treatment system such that water is treated without measuring any operating conditions. The controller can also control the operating conditions in closed-loop control so that any one or more operating parameters can be adjusted based on an operating condition measured by, for example, a sensor. The controller, or components or subsections thereof, may alternatively be implemented as a dedicated system or as a dedicated programmable logic controller (PLC) in a distributed control system.

In certain aspects, a controller, through one or more sensors, can monitor and/or measure a property of water in the treatment system. For example, the conductivity of water in a storage system, module current, the flow rate or water property of liquid flowing in a product stream, and the water properties of a feed stream can all be detected by one or more sensors and the data can be subsequently sent to a controller. In addition, the controller can adjust an operating parameter of one or more components of the water treatment system. For example, a controller can control the opening or closing of one or more valves in the process system.

One or more controllers may respond to error signals originating from the treatment system, and respond by interrupting or modifying one or more operating parameters related to the treatment system. For example, an error signal may prompt the controller to turn on or off or otherwise regulate flow to a water treatment device, such as an electrochemical water treatment device. An error signal may also prompt the controller to modify the treatment process to perform any one of a number of functions, for example, to engage in a cleaning, maintenance, and/or shutdown procedure. In addition, the controller may respond to one or more signals by reversing flow in the treatment process. For example, a signal could relay information related to the process and reflecting a certain period of elapsed time and/or a water conductivity value that is below a certain threshold; thereby prompting the controller to reverse the process flow.

The controller may include any suitable hardware and/or software that may be utilized to implement the control system. For example, one or more microcontrollers and microprocessors, including programmable devices and unprogrammable devices, or general-purpose microprocessors and/or memory configured to store data, program instructions for the general-purpose processing operations, and/or the methods and systems described herein may be included. Alternative types of controllers may include, for example, processors, digital signal processors, state machines, field programmable gate arrays, programmable logic devices, discrete circuitry, and the like.

The term “microcontroller” as used throughout the specification and claims, is used for the sake of simplicity and is meant to include any electronic device or circuit which is capable of comparing a signal generated by a sensor to a reference value or signal. The term “microcontroller” can include, but is not limited to, one or more microcontrollers, one or more microprocessors, and any circuit(s), device(s), or combinations thereof which are capable of achieving these objectives. In addition to arithmetic and logic elements of a general purpose microprocessor, the microcontroller may include features, such as read only, read write memory, and input/output interfaces. The methods and systems disclosed here may utilize a microprocessor with substitutable peripheral devices such as memory, timers and the like, which are intended to be encompassed within the term microcontroller. Therefore, a microcontroller refers to the microprocessor and any needed peripherals, inputs/outputs to perform the desired functions, such as measuring or monitoring, recording measurements into memory, comparing measurements, receiving electrical signals from a sensor, and sending instructions to another piece of process equipment, for example, a valve.

The control system can further comprise a communication system, for example, a remote communication device, for transmitting or sending measured operating conditions or operating parameter to a remote station.

In one or more embodiments, an RFID antenna can be used to provide positional and other information regarding the water treatment system, such as one or more water properties.

The RFID antenna senses the targeted information and an associated RFID antenna control processor can transmit the information to a system processor, thereby providing one method of in-line real-time process control. Non-limiting examples of suitable antennas include the Xbee brand manufactured by Digi International Inc.

One or more components of the water treatment system may be connected to a communication network that is operatively coupled to a control system. For example, sensors may be configured as input devices that are directly connected to the control system. Additionally, metering valves and/or pumps of the process system may be configured as output devices that are connected to the control system, and any one or more of the above may be coupled to another ancillary computer system or component so as to communicate with the control system over a communication network. Such a configuration permits one sensor to be located at a significant distance from another sensor or allows any sensor to be located at a significant distance from any subsystem and/or the controller, while still providing data therebetween.

One or more control systems can be implemented using one or more computer systems. The computer system may be, for example, a general-purpose computer such as those based on an Intel PENTIUM®-type processor, a Motorola PowerPC® processor, a Sun UltraSPARC® processor, a Hewlett-Packard PA-RISC® processor, or any other type of processor or combinations thereof. Alternatively, the computer system may include PLCs, specially-programmed, special-purpose hardware, for example, an application-specific integrated circuit (ASIC), or controllers intended for analytical systems.

In accordance with another embodiment of the systems and methods described herein, a controller regulates the operation of the treatment system by incorporating adaptive or predictive algorithms, which are capable of monitoring demand and water quality and adjusting the operation of any one or more components of the treatment system. For example, the controller may be predictive in anticipating higher demand for treated water during early morning hours in a residential application to supply water serving a showerhead. Predictive control models may be particularly useful where control is desired based upon particular system parameters that are impossible or difficult to detect. Further, in some embodiments, the control actions may be determined using a dynamic predictive model which may not only be adapted to control quality targets, but may also take cost considerations (for example, based on a cost function) into account. The control actions may be integrated with, for example, broadband and mobile access devices to provide remote interaction with the treatment system. For example, a user may interact with the control system through the use of a network device, such as a cellular phone.

In some embodiments, the control system can include one or more processors typically connected to one or more memory devices, which can comprise, for example, any one or more of a disk drive memory, a flash memory device, a RAM memory device, or other device for storing data. The one or more memory devices can be used for storing programs and data during operation of the treatment system and/or a control subsystem. For example, the memory device may be used for storing historical data relating to the parameters over a period of time, as well as current operating data. Software, including programming code that implements embodiments of the systems and methods disclosed here, can be stored on a computer readable and/or writeable nonvolatile recording medium, and then copied into one or more memory devices where it can then be executed by one or more processors. Programming code may be written in any of a plurality of programming languages, for example, ladder logic, Java, Visual Basic, C, C#, or C++, Fortran, Pascal, Eiffel, Basic, COBOL, Pearl, Python, and any variety of combinations thereof. Additionally, programming code may be taken from public domain open-source coding libraries, or from software copyrighted under a public licensing arrangement, such as a GNU General Public License, and modified for the instant purposes.

Components of a control system may be coupled by one or more interconnection mechanisms, which may include one or more busses, for example, between components that are integrated within a same device, and/or one or more networks, for example, between components that reside on separate discrete devices. The interconnection mechanism typically enables communication, for example, data and instructions, to be exchanged between components of the process system.

The control system can further include one or more input devices, for example, a keyboard, mouse, trackball, microphone, touch screen, and one or more output devices, for example, a printing device, display screen, or speaker. In addition, the control system may contain one or more interfaces that can connect to a communication network, in addition to, or as an alternative to the network that may be formed by one or more components of the control system.

In certain embodiments, a computer can be coupled to a server and to a plurality of different input devices. The input devices may include, for example, a wireless communication device (for example, a radio frequency identification (RFID) antenna), one or more sensors, a touch screen having a virtual keyboard, and one or more monitoring devices. In addition, the RFID antenna, any of the sensors, and/or the touch screen, may be configured to operate both as input devices and/or output devices. The touch screen is optional and may alternatively include other known input devices such as a keyboard, mouse, touch pad, joystick, remote control (either wireless or with a wire), track ball, mobile device, etc.

In certain embodiments, the control system includes a graphical user interface (GUI) to allow a user to monitor the process system, perform data entry functions, perform programming operations, and communicate with other users in a network. Process system data may be displayed by the GUI in one or more forms, for example, in graph form, so that a user or other personnel can readily monitor trends associated with various operating components in the process system. The GUI also enables a user to control or otherwise interact with the process system.

In certain non-limiting embodiments, a computer is wirelessly coupled to a server and an RFID antenna and one or more other sensors. The RFID antenna may receive input from an RFID device, such as a tag device, secured or otherwise in communication to one or more components of the process system. The RFID device can be programmed to include a wide range of information, and additional monitoring information collected during one or more water treatment cycles can be added to the RFID device. When the RFID device is in communication with the RFID antenna, any information programmed into the RFID device can be downloaded onto the computer and transferred to the server. The RFID device may also include an encryption device.

In certain non-limiting embodiments, radio frequency identification (RFID) is utilized to provide real-time detection of certain properties or conditions in a water treatment system. In certain embodiments, a plurality of inline identifying tag readers or optical sensors are configured to track or sense certain properties or conditions of the water as it is transported through the treatment system. The RFID may be combined with one or more additional sensors, such as a flowmeter. For example, an embedded tag may be placed in the cartridge of an ion exchange device and used in combination with a flowmeter to determine various properties or conditions, for example, the presence of the ion exchange resin, the usable volume remaining in the cartridge, and the number of days remaining before the cartridge is exhausted and needs to be replaced.

The control system can include one or more types of computer storage media such as readable and/or writeable nonvolatile recording medium in which signals can be stored that define a program to be executed by one or more processors. The storage or recording medium may be, for example, a disk or flash memory. In typical operation, the processor can cause data, such as code that implements one or more embodiments of the methods and systems disclosed here, to be read from the storage medium into a memory device that allows for faster access to the information by the one or more processors. The memory device is typically a volatile, random access memory such as a dynamic random access memory (DRAM), or static memory (SRAM), or any other suitable devices that facilitate information transfer both to and from the one or more processors.

The control system may have different access levels to control and monitor the process system. The control system may provide for different levels of user control over one or more components of the water treatment system. By providing multiple interfaces to the system with different levels of control, the integrity of the treatment system can be further protected. Subsets of control features can be provided through, for example, a touch screen or local computer. A low or very simple level of control may be exercised at the homeowner level, where process system operating parameters may be viewed, but little or no controlling interaction is allowed between the homeowner and the process system. In addition, an intermediate level of control or system access may be available for local service personnel who may need to review, monitor, or control one or more process systems in a certain geographic region. The highest level of control or system access may be reserved for a central office, which may be responsible for monitoring multiple process systems on a nation-wide basis.

The control system is configured to be able to provide different targets for one or more operating parameters in the water treatment system. For example, different users may prefer or require different conductivity levels in their respective product streams, or be limited by local laws as to the nature and volume of waste they are allowed to discharge to the environment. The operating parameters of the water treatment system may also be changed or adapted, for example, based on the time of day, the end use of the product stream, a user's preference, climate conditions, and the like. A user's preference may be based on user-input customizations or settings, for example, one user may prefer a product water stream with a hardness of around zero mg/L, whereas a second user may prefer water with a hardness of around 60 mg/L.

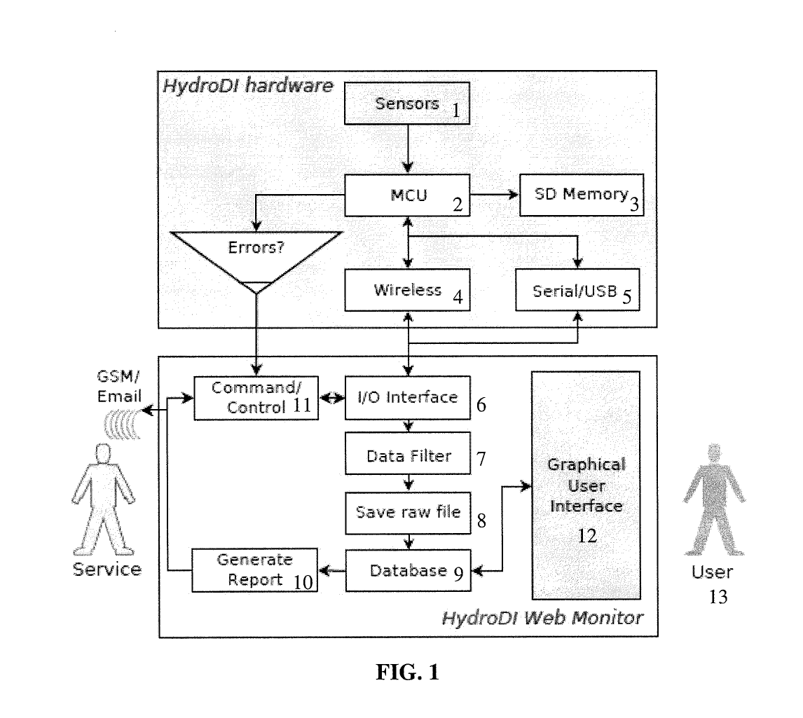

At least one embodiment of the systems and methods described herein is described with reference to FIG. 1.

FIG. 1 is a flow chart illustrating an exemplary control scheme in accordance with one or more embodiments of the systems and methods disclosed herein. Other components may be included in the control scheme depending upon the system design, the type of system controlled, the system control needs, and so forth. One or more sensors 1, as known in the art, may be positioned at desired locations within a water treatment process to detect process information, for example, one or more of various characteristics of the water to be treated, various characteristics of the treated water, and various system or unit operating conditions. Examples of suitable sensors include, but are not limited to, sensors to detect temperature, pH, conductivity, in-line microbial count, flow rate, module conductivity, and combinations thereof. The one or more sensors 1 are in communication with microcontroller 2, which may generate one or more signals or responses to one or more operating parameters in the treatment system. The signals are received by control computer 11 through I/O interface 6 and may be communicated any number of ways, for example, with a physical interface, such as serial port 5, and/or with a wireless data connection device 4. The I/O interface, as used here, is any device or program that is configured to identify and receive input signals and send output signals, and may be, for example, a wire or wireless interface. I/O interface 6 may be used to communicate with one or more peripheral devices including, for example, a server or other computer system, a network device, or a subsystem. I/O interface 6 may include, for example, a serial port, a parallel port, a Small Computer System Interface (SCSI), an IR interface, an RF interface, and/or a universal serial bus (USB) interface.

Control computer 11 may include any suitable processor, such as a microprocessor, a field programmable gate array, and so forth. Control computer 11 may carry out control functions and may perform model predictive control functions based upon knowledge of certain aspects of the process system. Control computer 11 may execute one or more model predictive control algorithms to develop values for a controlled variable. Such algorithms may be defined by one or more control models stored in a memory communicatively coupled to the control computer 11. The one or more control models may include a plurality of control models operating in cooperation to achieve a particular control objective. The control models may use desired variables, variable settings, set points, and so forth, as will be appreciated by those skilled in the art.

The control computer, based upon the control algorithm or algorithms, may output signals to microcontroller unit 2 that may be used to drive various components of the process system. The interface circuitry may include various driver circuits, amplification circuits, digital-to-analog conversion circuitry, and so forth. Based upon the process information received, the controller may determine appropriate control actions or outputs based on the variable relationships, constraints, and/or objectives defined by the control models. The controller may also include communications interface circuitry. By way of example, the communications interface circuitry may include networking circuitry configured to network the controller with other controllers that may be implemented in the control system, as well as with remote monitoring and control systems.

Control computer 11 runs a first program that maintains a serial connection to microcontroller unit 2 and writes incoming data to a file. On a periodic basis, for example, every 24 hours, the file is closed and stored, and a new one is created. The files are stored so that they are accessible from one or more remote computers. Graphical user interface 12 accesses the process system data and displays it locally and/or externally, for example, on a secure website. A second program functions as data filter 7, by periodically taking accumulated data in the file, sorting it for errors, and sending it to database 9. Non-limiting examples of errors include early carriage returns, nonsensical data, missing data, and the like. On a periodic basis, for example, every 24 hours, a third program uses the data from database 9 and performs analysis to generate a report 10 that summarizes the process system performance. The system performance report is stored locally and is also communicated to one or more remote clients, for example, service personnel, homeowners, and/or a monitoring station. The generated report and/or the contents therein may be communicated electronically to an external message delivery system, for example, an email server, an SMS text message or MMS message gateway, a Twitter or Facebook or other social media messaging system, one or more computer systems, and any other method for communicating data.

In certain embodiments, the control system includes a graphical user interface (GUI) 12 to allow a user 13 to perform a variety of functions, for example, monitor the process system, perform data entry functions, perform programming operations, and/or communicate with other users in a network. Process system data may be displayed by the GUI in one or more forms, for example, in graph form, so that user 13 or other personnel can readily monitor trends associated with various operating components in the process system. GUI 12 may also be capable of enabling a user to control or otherwise interact with the process system.

In one exemplary embodiment, a microcontroller is configured to instruct a valve to open or remain open when a sensor detects the presence of water (or desired fluid) or the absence of water. In a similar manner, when a sensor detects the presence or absence of water, the microcontroller may instruct a valve to close. The microcontroller may also instruct a valve to close upon the expiration of a programmed time.

Those skilled in the art should appreciate that the parameters and configurations described herein are exemplary and that actual parameters and/or configurations will depend on the specific application in which the various embodiments of the methods and systems described herein are used. Those skilled in the art should also recognize or be able to ascertain, using no more than routine experimentation, equivalents to the specific embodiments of the methods and systems described herein. It is, therefore, to be understood that the embodiments described herein are presented by way of example only and that, within the scope of the appended claims and equivalents, the invention may be practiced otherwise than as specifically described.

What is claimed is:

1. A method for monitoring and controlling a water treatment system comprising:
remotely reading a stream of measured data associated with the water treatment system;
performing an analysis of the measured data to determine one or more measured operating parameters associated with the water treatment system;
comparing the measured operating parameter to a target operating parameter;
producing an output response based on the comparison between the measured operating parameter to the target operating parameter; and
communicating the output response to at least one remote client electronically.

2-19. (canceled)

20. The method of claim 1, further comprising storing, on a storage device, an indication of the output response.

21. The method of claim 20, further comprising creating a database comprising the stored indication of the output response.

22. The method of claim 1, further comprising adjusting a flow rate of a water stream in the water treatment process based at least in part on the output response.

23. A method for monitoring the removal of unwanted species from a water stream in a water treatment system, the method comprising:
remotely reading at least one measured operating value related to the removal of unwanted species from the water stream;
comparing the at least one measured operating value to a target value;
generating an output response based on the comparison between the at least one measured operating value to the target value; and
transmitting the output response to at least one remote client.

24. The method of claim 23, wherein the at least one measured operating value is a concentration of unwanted species in the water stream.

25. The method of claim 24, wherein the water stream is a product water stream.

26. The method of claim 25, wherein the product water stream has been treated by an electrochemical water treatment device.

27. The method of claim 23, wherein the at least one measured operating value is a remaining cartridge capacity in at least one treatment device.

28. The method of claim 23, further comprising controlling at least one operating parameter of the water treatment system by the at least one remote client based on the output response.

29. A system for monitoring a water treatment process comprising:
a remote interface device in communication with at least one component of the water treatment process; and
a controller in communication with the remote interface device and configured to:
read data associated with the water treatment process;
perform an analysis of the data associated with the water treatment process to determine one or more parameters associated with the water treatment process;
compare the one or more parameters associated with the water treatment process to one or more desired parameters; and
produce an output response based on the comparison of the one or more parameters associated with the water treatment process to the desired parameters.

30. The system of claim 29, further comprising at least one sensor configured to measure the data associated with the water treatment process.

31. The system of claim 29, further comprising at least one treatment device associated with the water treatment process.

32. The system of claim 29, wherein the at least one treatment device includes at least one replaceable cartridge.

33. The system of claim 29, wherein the output response is a remaining cartridge capacity for the at least one replaceable cartridge.
